(19) United States
(12) Patent Application Publication
(10) Pub. No.: US 2022/0105132 A9
(43) Pub. Date: Apr. 7, 2022

(54) Method for Producing an Exosome Containing Agent for Metastasis Suppression

(71) Applicant: Vita Motus AG, Wattwil (CH)

(72) Inventors: Aleksandr Abramov, Moscow (RU); Alisa Petkevic, Moscow (RU); Vadim Pospelov, Kolomna (RU)

(21) Appl. No.: 16/668,581

(22) Filed: Oct. 30, 2019

(30) Foreign Application Priority Data
Oct. 31, 2018 (RU) 2018138338
Aug. 5, 2019 (CH) 00987/19

(51) Int. Cl. A61K 35/13; A61K 9/127; C12N 5/09

(52) U.S. Cl. CPC A61K 35/13; A61K 9/0019; C12N 5/0693; A61K 9/1271

(57) ABSTRACT

Disclosed herein is a method for producing an exosome containing therapeutic agent, including: separating tumor cells from a biomaterial sample of a subject; treating the separated tumor cells in a culture media at 37° C. and at 5% CO2 for 24-72 h; separating an exosome composition from the culture media; and dissolving the separated exosome composition in a phosphate buffer to obtain the exosome containing therapeutic agent; characterized in that the culture media comprises either (i) a 1:1 mixture of DMEM/F12 (Dulbecco's Modified Eagle Medium: Nutrient Mixture F-12) and HEPES (4-(2-hydroxyethyl)-1-piperazineethanesulfonic acid), 2 mmol/mL or 3.65 mg/10 mL of L-glutamine, 100 units/mL of penicillin, 100 μg/mL of streptomycin, and 10 wt % of fetal bovine serum; or (ii) 79 wt % of RPMI, 20 wt % of fetal bovine serum and 1 wt % of streptomycin.

### CROSS-REFERENCE TO RELATED APPLICATION

This application claims priority to Switzerland Patent Application No. 00987/19 filed Aug. 5, 2019, the disclosure of which is hereby incorporated by reference in its entirety.

### FIELD OF DISCLOSURE

The present invention is directed to a method for producing an exosome containing therapeutic agent for metastasis suppression and a method for treatment with an exosome containing therapeutic agent.

### BACKGROUND, PRIOR ART

RO 2593003 C2 discloses a method of tumor immunotherapy based on administration of exosome-containing medication to the patient, in which patients with cancer are treated with exosomes, isolated from cell culture K562/i-S9, which does not allow to obtain specific exosomes, so that it is not possible to achieve a high efficiency of the immune response, in contrast to the exosomes, produced by the patient's own tumor cells—which contain specific antigens, similar to the antigens of tumor cells, which allow to achieve a highly effective immune response, in consequence of which the known method is not effective enough.

### SUMMARY OF DISCLOSURE

It is known that exosomes are involved in various stages of immune reactions, in particular, exosomes secreted by B-cells carry on their surface receptors of MHC II (the main histocompatibility complex II), co-stimulating and adhering molecules, which indicates their ability to directly stimulate CD4 positive T-cells. [B lymphocytes secrete antigen-presenting vesicles. Raposo G, Nijman H W, Stoorvogel W, Liejendekker R, Harding C V, Melief C J, Geuze H J J Exp Med. 1996 Mar. 1; 183(3):1161-72.] To enhance this effect, exosomes are concentrated, for example, by magnetic beads and loaded with peptides. Along with the ability to activate CD4 positive cells, exosomes obtained from dendritic cells and pooled with peptides of processed tumor antigens are able to activate tumor-specific cytotoxic lymphocytes and thereby inhibit tumor growth. [Eradication of established murine tumors using a novel cell-free vaccine: dendritic cell-derived exosomes. Zitvogel L, Regnault A, Lozier A, Wolfers J, Flament C, Tenza D, Ricciardi-Castagnoli P, Raposo G, Amigorena S Nat Med. 1998 May; 4(5):594-600.]

Thus, exosomes, secreted by tumor cells, also act as immune system activators, but with the course of the tumor process there are transformations, allowing atypical cells to avoid the action of immunocompetent cells, one of these transformations is the inability of lymphocytes to perceive exosomes as carriers of tumor's genetic material. When using exosomes per os, obtained from the patient, there is contact between the cells of the immune system and exosomes, synthesized by the tumor cells, which can lead to activation of antitumor immunity.

It is a general object of the invention to provide a further developed therapeutic agent for metastasis suppression. In favorable embodiments a therapeutic agent with increased effectiveness is provided.

In a first aspect of the invention, the general object is achieved by a method for producing an exosome containing therapeutic agent, comprising the steps:
Separating tumor cells from a biomaterial sample of a subject;
Treating the separated tumor cells in a culture media at 37° C. and at 5% CO₂ for 24-72 h or treating the separated;
Separating an exosome composition from the culture media;
Dissolving the separated exosome composition in a phosphate buffer to obtain the exosome containing therapeutic agent;
   characterized in that the culture media comprises either
(i) a 1:1 mixture of DMEM/F12 (Dulbecco's Modified Eagle Medium: Nutrient Mixture F-12, according to Dulbecco, R. & Freeman, G. (1959) Virology 8, 396 and Smith et al. (1960) Virology 12, 185) and HEPES (4-(2-hydroxyethyl)-1-piperazineethanesulfonic acid), 2 mmol/mL or 3.65 mg/10 mL L-glutamine, 100 units/mL penicillin, 100 μg/mL streptomycin, 10 wt % fetal bovine serum; or
(ii) 79 wt % RPMI, 20 wt % fetal bovine serum and 1 wt % streptomycin.

As the skilled person understands, RPMI, also known as RIMO 1640, is a growth medium used in cell culture (Atlas R M, Snyder J W (2006). Handbook of Media for Clinical Microbiology (2nd ed.). Boca Raton, Fla.: CRC Press. p. 427.)

It is understood that the method for producing an exosome containing therapeutic agent and in particular step of separating tumor cells, is performed in vitro.

Typically, separating tumor cells from a biomaterial sample of a subject is achieved by centrifugation.

In some embodiments, dissolving the separated exosome composition in a phosphate buffer is performed such that a ratio of 1:5 to 1:20 of separated exosome composition to phosphate buffer is obtained.

In further embodiments, the biomaterial sample consists of venous blood. Alternatively, it is also possible to use the subject's peripheral blood or tumor tissue obtained intraoperatively, or an ascetic/pleuritic liquid of metastatic genesis.

In preferred embodiments, separating the tumor cells comprises:
a. exposing the biomaterial to an EDTA anticoagulant;
b. dilution by a factor of 2 with Hanks salt solution;
c. application of the diluted mixture to Ficoll-Urografin (F-U)□solution with a density of 1.077 g/cm3 in a ratio of in a ratio of 1:4, followed by centrifugation for 15 min at 2000 rpm;
d. collecting a generated interphase ring;
e. diluting the interphase ring with a phosphate buffer solution in a ratio of 1:15 to 1:20 and subsequent centrifugation for 10 min with 1500 rpm to provide a sediment;
f. collecting the sediment, adding lysing solution and maintaining the collected sediment and the lysing solution at 15-25° C. for 10 min;
g. mixing with a phosphate buffer with 0.9 M CaCl2 solution
h. collecting the tumor cells.

The advantage of this method of isolation of tumor cells is the ability to obtain the most quantitatively possible pool of tumor cells while maintaining their viability and sterility. At the same time, this method allows, with minimal losses for tumor cells, to get rid of cells that do not fall into the pool of interest, such as red blood cells, leukocytes and, partially, platelets. The use of certain types of enzymes, such as accutase, minimizes damage to cell surface receptors and antigenic determinants. Centrifugation conditions are selected in such a way as to allow to differentiate debris (cell fragments and decomposition products) with exosomes from cells, on the stages of work with a supernatant containing a precipitate with cell fragments, decomposition products and exosomes, the described work conditions allow to obtain from the entire sediment a fraction with the highest content of exosomes.

In some embodiments, the step of separating the exosome composition from the culture media further comprises a differential centrifugation.

In further embodiments, the differential centrifugation is performed for 10 min at 3000 rpm, followed by collecting a generated first supernatant and centrifuging the first supernatant for 30 min at 10 000 rpm, followed by collecting a generated second supernatant and ultra-centrifuging the second supernatant for 2 h at 100 000 rpm, wherein after each centrifuging step, an exosome composition is separated.

According to a second aspect, the invention is directed to a method of treating metastasis generation in a subject, comprising:
Obtaining a biomaterial sample of the subject;
Separating tumor cells from the biomaterial sample;
Treating the separated tumor cells in a culture media at 37° C. and at 5% CO₂ for 24-72 h or treating the separated;
Separating an exosome composition from the culture media;
Dissolving the separated exosome composition in a phosphate buffer to obtain the exosome containing therapeutic agent;
Administering the therapeutic agent to the subject intravenously; characterized in that the culture media comprises either
(i) a 1:1 mixture of DMEM/F12 and HEPES, 2 mmol/mL or 3.65 mg/10 mL L-glutamine, 100 units/mL penicillin, 100 μg/mL streptomycin, 10 wt % fetal bovine serum; or
(ii) 79 wt % RPMI, 20 wt % fetal bovine serum and 1 wt % streptomycin.

In some embodiments, dissolving the separated exosome composition in a phosphate buffer is performed such that a ratio of 1:5 to 1:20 of separated exosome composition to phosphate buffer is obtained.

In further embodiments, the biomaterial sample consists of venous blood. Alternatively, it is also possible to use the subject's peripheral blood or tumor tissue obtained intraoperatively, or an ascetic/pleuritic liquid of metastatic genesis.

In preferred embodiments, separating the tumor cells comprises:
a. exposing the biomaterial to an EDTA anticoagulant;
b. dilution by a factor of 2 with Hanks salt solution;
c. application of the diluted mixture to diatrizaoate (urografin) solution with a density of 1.077 g/cm³ in a ratio of in a ratio of 1:4, followed by centrifugation for 15 min at 2000 rpm;
d. collecting a generated interphase ring;
e. diluting the interphase ring with a phosphate buffer solution in a ratio of 1:15 to 1:20 and subsequent centrifugation for 10 min with 1500 rpm to provide a sediment;
f. collecting the sediment, adding lysing solution and maintaining the collected sediment and the lysing solution at 15-25° C. for 10 min;
g. mixing with a phosphate buffer with 0.9 M CaCl₂ solution
h. collecting the tumor cells.

In some embodiments, the step of separating the exosome composition from the culture media further comprises a differential centrifugation.

In further embodiments, the differential centrifugation is performed for 10 min at 3000 rpm, followed by collecting a generated first supernatant and centrifuging the first supernatant for 30 min at 10 000 rpm, followed by collecting a generated second supernatant and ultra-centrifuging the second supernatant for 2 h at 100 000 rpm, wherein after each centrifuging step, an exosome composition is separated.

Exemplary embodiments

Patient, 14 years old, student, medical history without abnormalities, acute B-lymphoblastic leukemia, cytogenetic variant t (1;19)(q23;p13), E2A/PBX1. Blasts in bone marrow aspirate (CD19+, CD79+, CD22cyt+, TdT+, HLADR+) 17.5%, the leukocytic component exceeds the upper limit, neutrophilic metamyelocytes are increased the most (up to 67%), myeloblasts are not defined. In the haemogram there are blast cells 9%, metamyelocytes 3%, erythropenia up to 2.9*10\hat{ }12/L, leukopenia 2.3*10\hat{ }9/L. At the maintenance therapy stage, along with 6-mercaptopurine, exosomes obtained from autologous tumor cells were administered per os 2 times a week.

Two months after drug administration and on maintenance therapy, the patient demonstrated decrease in leukopenia from 2.3*10\hat{ }9/L to 3.1*10\hat{ }9/L, which indicates a gradual increase of the proportion of mature leukocytes in the bloodstream; the number of blasts in the bone marrow aspirate decreases from 17.5% to 14%, blast cells are not determined in the haemogram. Among the immunological parameters, there is an increase in the cytotoxic activity of NK (natural killer) cells from 65% before drug administration to 78% after drug administration (with the ratio of effector cells to target cells 1:5). These changes support the improvement of the patient's condition and stabilization of the process.

The tumor cells were isolated from the patient's venous blood, the venous blood is drawn into a test tube with an anticoagulant EDTA, then the resulting blood is diluted 2 times with the Hanks' solution, then it is layered on a fikoll-urographin solution with a density of 1.077 g/cm2 at a ratio of 1:4, then centrifuged at 4° C. at 2000 rpm for 15 minutes, then the interphase ring formed during centrifugation is collected and phosphate-buffer saline buffer is added at a ratio of 1:15 and then subjected to centrifugation at 1500 rpm for 10 minutes, then the precipitate is isolated and 2 ml of the lysing solution is added and the resulting mixture is kept at 15° C. for 10 minutes, then the phosphate-buffer saline buffer is added to the mixture at a ratio of 1:10 and stirred, then it is centrifuged at 1500 rpm for 10 minutes, then the supernatant is poured out and 4 ml of phosphate-buffer saline buffer is added to the precipitate, further containing 0.9 M CaCl2 and stirred, then tumor cells are isolated from the obtained suspension in the form of precipitate by passing it through a microtubule, then the resulting precipitate with tumor cells is cultured in a culture medium of the following composition: 79% RPMI, 20% FBS, 1% solution of penicillin streptomycin, under conditions t=37° C. and 5% CO2 for 72 hours, with supernatant being isolated every 24 hours, and the culture medium of the following composition added to the remaining precipitate: 79% RPMI, 20% FBS, 1% solution of penicillin streptomycin, in the volume of the isolated supernatant. An isolation of exosome-containing sediment from the resulting culture medium is carried out by differential centrifugation, namely: the resulting culture medium is centrifuged at 3000 rpm for 10 min., then the resulting supernatant is collected and further centrifuged at 10,000 rpm for 30 minutes, then the resulting supernatant is collected and ultracentrifugation at 100,000 rpm for 2 hours is performed, then the formed precipitate is isolated.

1.1. To create a tumor model aimed at studying the processes of metastasis and activation of the immune response, immunocompetent male mice of the C57BL/6 line were selected at the age of no more than 4 weeks., n=27. 1×105 cells of murine melanoma line F10/B16 in phosphate buffered saline solution (PBS, 100 000 cells/0.1 ml PBS) were injected into the tail vein of all animals. The process of metastasis occurred during 8 days to the moment of exposure.

1.2. To obtain a preparation, the cells of the murine melanoma of the F 10/B16 line were thawed and cultivated for 72 h to reach the amount of 6 000 000, after that they were centrifuged at 3000 rpm, the supernatant was transferred to the Customer to prepare the Preparation.

1.3. The animals were divided into 3 groups: No. 1 (i.v. injection), No. 2 (oral administration), No. 3 (control).

1.4. Mice were kept in standard vivarium conditions with free access to briquetted food and water. The Preparation was administered on the 8-th and 11-th day from the date of inoculation:
in group 1, 100 μg of Preparation was injected into the tail vein
in group 2, 100 μg of Preparation was administered per os
in group 3, 100 μg of pure PBS 9 without Preparation) was injected into the tail vein The slaughter was conducted by the method of cervical dislocation 16 days after inoculation of tumor cells. In order to assess the metastatic process, a visual assessment of the presence and number of metastases in the liver was carried out and its mass was measured. Statistical significance was assessed using a one-way variance analysis in the program SPSS 22.
Average number of liver metastases in group No. 1: 5.5+−0.5. Average liver mass: 1.2+−0.1
Average number of liver metastases in group No. 2: 3.8+−0.3. Average liver mass: 1.1+−0.2
Average number of liver metastases in group No. 3: 9.0+−2.1. Average liver mass: 1.4+−0.2

Differences in the number of metastases in the observation groups from the control significantly (p<0.05). The difference in the number of metastases between groups 1 and 2 on the verge of significance (p<0.1). Liver mass in the observation and control groups differed insignificantly (p>0.05).

In order to assess the activation of the immune system, splenocytes were isolated from animal spleen, in which the percentage of cytotoxic T-lymphocytes CD8+ was determined by the method of flow cytometry. With this purpose, after isolation, the splenocytes were washed in PBS and incubated for an hour at 4C with antiCD8 antibodies, with further analysis on the flow cytofluorometer Becton Dickinson or Beckman Coulter Life Sciences.

The average percentage of CD8+ T-lymphocytes in group No. 1: 27%

The average percentage of CD8+ T-lymphocytes in group No. 2: 36%

The average percentage of CD8+ T-lymphocytes in group No. 2: 17%

The values “observation-control” differ significantly (p<0.05). The difference between groups 1 and 2 significantly (p<0.05).

Oral administration of the Preparation inhibits the process of metastasis in a more pronounced way compared to intravenous injection. Similar observations were noted during the analysis of the immune system activation degree. The process may be related to macrophage-mediated antigen presentation of the tumor to T-killers.

1. A method for producing an exosome containing therapeutic agent, comprising:
Separating tumor cells from a biomaterial sample of a subject;
Treating the separated tumor cells in a culture media at 37° C. and at 5% CO₂ for 24-72 hours;
Separating an exosome composition from the culture media;
Dissolving the separated exosome composition in a phosphate buffer to obtain the exosome containing therapeutic agent;
wherein the culture media comprises either:
(i) a 1:1 mixture of DMEM/F12 and HEPES, 2 mmol/mL or 3.65 mg/10 mL of L-glutamine, 100 units/mL of penicillin, 100 μg/mL of streptomycin, and 10 wt % of fetal bovine serum; or
(ii) 79 wt % of RPMI, 20 wt % of fetal bovine serum and 1 wt % of streptomycin.

2. The method of claim 1, wherein dissolving the separated exosome composition in a phosphate buffer is performed such that a ratio of 1:5 to 1:20 of separated exosome composition to phosphate buffer is obtained.

3. The method according to claim 1, wherein the biomaterial sample comprises venous blood.

4. The method according to claim 1, wherein the biomaterial sample comprises at least one of the subject's peripheral blood, the subject's tumor tissue obtained intraoperatively, or an ascetic/pleuritic liquid of metastatic genesis.

5. The method according to claim 1, wherein separating the tumor cells comprises:
exposing the biomaterial to an EDTA anticoagulant;
dilution by a factor of 2 with Hanks salt solution;
application of the diluted mixture to Ficoll-Urografin (F-U) solution with a density of 1.077 g/cm3 in a ratio of in a ratio of 1:4, followed by centrifugation for 15 min at 2000 rpm;
collecting a generated interphase ring;
diluting the interphase ring with a phosphate buffer solution in a ratio of 1:15 to 1:20 and subsequent centrifugation for 10 min with 1500 rpm to provide a sediment;
collecting the sediment, adding lysing solution and maintaining the collected sediment and the lysing solution at 15-25° C. for 10 min;
mixing with a phosphate buffer with 0.9 M CaCl2 solution; and
collecting the tumor cells.

6. The method according to claim 1, wherein the step of separating the exosome composition from the culture media further comprises a differential centrifugation.

7. The method of claim 6, wherein the differential centrifugation is performed for 10 min at 3000 rpm, followed by collecting a generated first supernatant and centrifuging the first supernatant for 30 min at 10 000 rpm, followed by collecting a generated second supernatant and ultra-centrifuging the second supernatant for 2 h at 100 000 rpm, wherein after each centrifuging step, an exosome composition is separated.

8. A method of treating metastasis generation in a subject, comprising:
Obtaining a biomaterial sample of the subject;
Separating tumor cells from the biomaterial sample;
Treating the separated tumor cells in a culture media at 37° C. and at 5% CO2 for 24-72 h or treating the separated;
Separating an exosome composition from the culture media;
Dissolving the separated exosome composition in a phosphate buffer to obtain the exosome containing therapeutic agent; and
Administering the therapeutic agent to the subject intravenously;
wherein the culture media comprises either:
a 1:1 mixture of DMEM/F12 and HEPES, 2 mmol/mL or 3.65 mg/10 mL of L-glutamine, 100 units/mL of penicillin, 100 μg/mL of streptomycin, and 10 wt % of fetal bovine serum; or 79 wt % of RPMI, 20 wt % of fetal bovine serum and 1 wt % of streptomycin.

9. The method of claim 8, wherein dissolving the separated exosome composition in a phosphate buffer is performed such that a ratio of 1:5 to 1:20 of separated exosome composition to phosphate buffer is obtained.

10. The method according to claim 8, wherein the biomaterial sample comprises venous blood.

11. The method according to claim 8, wherein the biomaterial sample comprises venous blood.

12. The method according to claim 8, wherein separating the tumor cells comprises:
exposing the biomaterial to an EDTA anticoagulant;
dilution by a factor of 2 with Hanks salt solution;
application of the diluted mixture to Ficoll-Urografin (F-U) solution with a density of 1.077 g/cm3 in a ratio of in a ratio of 1:4, followed by centrifugation for 15 min at 2000 rpm;
collecting a generated interphase ring;
diluting the interphase ring with a phosphate buffer solution in a ratio of 1:15 to 1:20 and subsequent centrifugation for 10 min with 1500 rpm to provide a sediment;
collecting the sediment, adding lysing solution and maintaining the collected sediment and the lysing solution at 15-25° C. for 10 min;
mixing with a phosphate buffer with 0.9 M CaCl2 solution; and
collecting the tumor cells.

13. The method according to claim 8, wherein the step of separating the exosome composition from the culture media further comprises a differential centrifugation.

14. The method of claim 13, wherein the differential centrifugation is performed for 10 min at 3000 rpm, followed by collecting a generated first supernatant and centrifuging the first supernatant for 30 min at 10 000 rpm, followed by collecting a generated second supernatant and ultra-centrifuging the second supernatant for 2 h at 100 000 rpm, wherein after each centrifuging step, an exosome composition is separated.
